(19) United States
(12) Patent Application Publication
(10) Pub. No.: US 2024/0009165 A1
(43) Pub. Date: Jan. 11, 2024

(54) GRISEOFULVIN AS AN ADJUNCT DRUG FOR THE TREATMENT OF CEREBRAL AND SEVERE MALARIA

(71) Applicant: INSTITUTE OF LIFE SCIENCES, Odisha Bhubaneswar (IN)

(72) Inventors: Viswanathan Arun NAGARAJ, Bhubaneswar (IN); Govindarajan PADMANABAN, Bangalore (IN); Manjunatha Chandana SHETTY, Bhubaneswar (IN); Aditya ANAND, Bhubaneswar (IN); Sourav GHOSH, Bhubaneswar (IN)

(21) Appl. No.: 18/253,744

(22) PCT Filed: Nov. 15, 2021
(86) PCT No.: PCT/IN2021/051071

§ 371 Date: May 19, 2023

(30) Foreign Application Priority Data
Nov. 23, 2020 (IN) 202031050934

(51) Int. Cl. A61K 31/343; A61K 31/357; A61K 31/135; A61K 31/4709; A61K 31/4706; A61K 31/635; A61K 31/496; A61P 33/06

(52) U.S. Cl. CPC A61K 31/343; A61K 31/357; A61K 31/135; A61K 31/4709; A61K 31/4706; A61K 31/635; A61K 31/496; A61P 33/06

(57) ABSTRACT

Malaria remains a major concern of morbidity and mortality. Despite treating infected individuals with artemisinin-based combination therapies (ACTs), cerebral and severe malaria cause enormous mortality in children and adults. The only treatment option is parenteral administration of artemisinin or quinine with supportive symptomatic therapies. However, the fatality rate remains high despite parasite clearance emphasizing the need of adjunct therapy to prevent malaria mortality. In this invention, heme synthesized in the malaria parasite is shown to be associated with parasite virulence, disease severity and cerebral pathogenesis. Parasite heme enhances the formation of hemozoin—a parasite molecule associated with cerebral and severe malaria. Griseofulvin prevents experimental cerebral malaria (ECM) by inhibiting parasite heme synthesis and hemozoin formation, without affecting parasite growth. Griseofulvin exhibits synergy with arteether—the principal component of ACTs and prevents ECM. Griseofulvin is claimed as an adjunct drug for ACTs and other antimalarials to prevent malaria mortality.

### FIELD OF THE INVENTION

This field relates to the treatment of malaria patients with griseofulvin as an adjunct drug along with artemisinin-based combination therapies (ACTs) or artemisinin derivatives or other antimalarials to prevent cerebral malaria and severe malaria.

### BACKGROUND AND PRIOR ARTS OF THE INVENTION

Malaria remains a major concern of morbidity and mortality, especially with the emerging parasite resistance to artemisinin-based combination therapies (ACTs) and mosquito resistance to insecticides. According to World Health Organization (WHO), 229 million cases and 409,000 malaria deaths occurred in 2019. Of the five Plasmodium species causing human malaria, Plasmodium falciparum (Pf) is the deadliest one responsible for more than 90% of the infections (1,2). The clinical manifestations of Pf malaria vary from mild (uncomplicated malaria) to severe (complicated malaria). Uncomplicated malaria is characterized by fever, headache, nausea, chills and mild anemia, with no clinical symptoms or laboratory findings related to organ dysfunctions. Complicated malaria is categorized by the existence of at least one criterion of disease severity that includes respiratory distress, metabolic acidosis, pulmonary edema, severe anemia, jaundice, renal failure or neurological complications like impaired consciousness, convulsions etc. The typical outcome of severe malaria is multi-organ failure and/or cerebral malaria (CM) of which, CM is the most severe neurological complication with high mortality (3-6). About one-third of the patients recovering from CM show long-term neurocognitive impairments. The only treatment option for CM is parenteral administration of artemisinin derivatives or quinine with supportive symptomatic therapies. However, the fatality due to CM remains high despite the parasite clearance (7-9). Therefore, it becomes important to understand the molecular mechanisms underlying CM pathogenesis for developing adjunct therapies. Further, the mortality due to cerebral and severe malaria happens despite parasite clearance and treating the patients with ACTs. Therefore, there is a need to develop new adjunct therapies that can prevent the mortality due to malaria.

The mortality due to cerebral and severe malaria is high and occurs despite providing ACTs—the existing antimalarial treatment regimen recommended by World Health Organization (WHO). The last resort is parenteral (intravenous/intramuscular) administration of quinine or artesunate, which are just antimalarials clearing the parasite, along with supportive symptomatic therapies. This invention specifically claims griseofulvin as an adjunct synergetic drug to treat cerebral and severe malaria, and not as a general adjunct antimalarial since it does not act like an antimalarial inhibiting the parasite growth. Griseofulvin acts by inhibiting hemozoin (Hz) formation, the known parasite inflammatory molecule underlying cerebral and severe malaria onset. The conceptual novelty of this invention is that griseofulvin can serve in as an adjunct drug by acting at a different target—heme synthesis, along with the existing antimalarials, providing synergy to prevent cerebral and severe malaria. The inclusion of griseofulvin in the existing ACTs can serve as a new product and therapeutic combination for preventing cerebral and severe malaria complications.

Cerebral pathology arises due to a complex interplay of molecular events triggered by various host- and parasite-derived factors. The synchronous growth of the asexual parasites in red blood cells (RBCs) and the associated schizogony result in the release of pathogen-associated molecular patterns (PAMPs) such as glycosylphosphatidylinositiol, parasite DNA and RNA, and Hz, and danger-associated molecular patterns such as uric acid, heme and microvesicles. Hz and its precursor heme play a central role in CM pathogenesis (10-14). The asexual stage parasites internalize host hemoglobin (Hb) by endocytosis and digest it in the acidic food vacuole (FV). The toxic free heme released during this process is detoxified into Hz, through a biocrystallization process catalysed by heme detoxification protein (HDP). Although the rate of HDP-mediated Hz formation is much higher, autocatalytic, histidine rich protein (HRP)-mediated and lipid-driven mechanisms for Hz formation have also been described. There is a positive correlation of Hz released into the circulation and phagocytosed by the circulating phagocytic cells with disease severity in children and adults. Similarly, plasma free heme is associated with disease severity (15-19). Free heme is extremely cytotoxic to endothelial cells and it can increase the expression of adhesion molecules, induce NLRP3 inflammasome and IL-1β secretion, and activate polymorphonuclear cells. Our current understanding on CM comes from a few post-mortem studies of human CM (HCM) and a large number of experimental CM (ECM) studies performed in mouse models. There were earlier discrepancies on the extent of using ECM as a model for HCM, especially in the context of the sequestration of parasitized RBCs (pRBCs) in brain microvasculature. HCM is characterized by strong cytoadherence and dense sequestration of large numbers of pRBCs that can obstruct substantial lengths of cerebral microvasculature, in contrast to irregular distribution of pRBCs in ECM that seem to be mechanically trapped. However, several studies in recent years have suggested significant analogies between HCM and ECM in terms of cerebral and neurovascular pathology. There is a consensus on increased permeability of blood-brain barrier (BBB), brain capillary occlusions, parasitized-red blood cells (pRBCs) accumulation in brain microvasculature, leukocyte infiltration, and endothelial activation with dysregulated inflammation and aberrant host-immune responses. All these lead to BBB disruption, intracerebral hemorrhages, ischemia, edema, increased intracranial pressure, axonal damage and demyelination, culminating in the dysfunction of central nervous system (20-25).

Malaria parasite synthesizes heme de novo despite the ability of asexual stages to access the host Hb-heme. The parasite heme pathway is compartmentalized in mitochondrion, apicoplast and cytosol, and heme is eventually synthesized in the mitochondrion. Earlier study with P. berghei (Pb) aminolevulinate synthetase (ALAS) and ferrochelatase (FC) knockouts (KOs) generated for the first and last enzymes, had demonstrated that the parasite pathway is dispensable for the asexual blood stage development, but essential for the development of sporozoites in the mosquitoes and pre-erythrocytic stages in the liver. Moreover, the asexual FCKO parasites can utilize host heme for cytochrome biogenesis to support pyrimidine metabolism. Subsequent studies carried out in Pf using the KO parasites generated for ALAS, FC, apicoplast-localized porphobilinogen deaminase and cytosol-localized coproporphyrinogen oxidase, had confirmed these findings. Although de novo heme synthesis is non-essential for asexual stages and the KO parasites can acquire heme and/or its precursors from host RBCs and import some of the host enzymes, the parasite enzymes are expressed and heme synthesis occurs in the asexual stages (26-29). This invention utilized the well-established in vivo Plasmodium berghei (rodent parasite)-infected mouse model for cerebral malaria and show that the parasite de novo heme pathway has a physiological relevance in the asexual stages and it is associated with disease pathogenesis by regulating Hz formation. The small amount of biosynthetic heme synthesized in the asexual stages can regulate the conversion of a large amount of Hb-heme into Hz, since biosynthetic heme is essential for the functional integrity of FV in which Hz formation is taking place (30). Griseofulvin—a FDA-approved antifungal drug, isolated from Penicillium griseofulvum, is used to cure tinea infections. It is known to interact with fungal microtubules and disrupts spindle assembly leading to mitotic arrest. In humans, griseofulvin dosage is given to the extent of 1000 mg/day in adults and 10 mg/kg/day in children for several weeks. Griseofulvin is also known to inhibit FC—the terminal enzyme of the heme-biosynthetic pathway, by generating N-methyl protoporphyrin IX (NMPP) through the action of cytochrome P450 enzymes (31-33). Until now, griseofulvin has been proposed or tested only as an antimalarial for inhibiting parasite growth. It turned out to be an ineffective antimalarial since it does not inhibit in vivo parasite growth (34-36). In this invention, a new technical advancement of griseofulvin is provided as being useful for preventing cerebral and severe malaria, although it does not inhibit parasite growth. Our claim is novel and new, and in none of the existing literature or patents, its ability to prevent cerebral malaria or severe malaria has been proposed or shown. The present invention claims that inhibiting parasite heme synthesis and Hz formation in the FV by griseofulvin can serve as a new synergistic adjunct therapy with the existing frontline therapies such as ACTs and other antimalarials to prevent CM and severe malaria in humans.

WO 1996016664A1 talks about usage of griseofulvin against protozoan infection, however, claims for the use of adenosine derivative and deaminase inhibitor for fungal and parasitic infections either alone or in combination with existing treatment modalities. Griseofulvin usage is mentioned as one of the antifungals.

Ramakrishnan G. et al; (Exploring anti-malarial potential of FDA approved drugs: an in silico approach.) mention griseofulvin as a potential anti-malarial drug in Table 3. This publication and the table provided list FDA approved drugs that can be explored for their antimalarial potential.

As already mentioned, griseofulvin does not inhibit parasite growth and this invention is not claiming griseofulvin as an antimalarial to inhibit parasite growth. Rather, this invention is for its ability to prevent cerebral and severe malaria, and therefore, griseofulvin can serve as an adjunct drug to prevent malaria mortality.

### FURTHER REFERENCES

1) World Health Organization. World Malaria Report (2020). https://www.mmv.org/sites/default/files/uploads/docs/publications/World Malaria R eport 2020.pdf
2) Global report on insecticide resistance in malaria vectors: 2010-2016. (2018). World Health Organization. https://apps.who.int/iris/bitstream/handle/10665/272533/9789241514057-eng.pdf?ua=1
3) Grobusch M P, Kremsner P G. Uncomplicated malaria. Curr Top Microbiol Immunol. 2005; 295:83-104.
4) Conroy A L, Datta D, John C C. What causes severe malaria and its complications in children? Lessons learned over the past 15 years. BMC Med. 2019; 17:52.
5) Varo R, Crowley V M, Sitoe A, Madrid L, Serghides L, Kain K C, Bassat Q. Adjunctive therapy for severe malaria: a review and critical appraisal. Malar J. 2018; 17:47.
6) Dunst J, Kamena F, Matuschewski K. Cytokines and chemokines in cerebral malaria Pathogenesis. Front Cell Infect Microbiol. 2017; 7:324. doi: 10.3389/fcimb.2017.00324.
7) John C C, Bangirana P, Byarugaba J, Opoka R O, Idro R, Jurek A M, Wu B, Boivin M J. C M in children is associated with long-term cognitive impairment. Pediatrics. 2008; 122:e92-9.
8) John C C, Panoskaltsis-Mortari A, Opoka R O, Park G S, Orchard P J, Jurek A M, Idro R, Byarugaba J, Boivin M J. Cerebrospinal fluid cytokine levels and cognitive impairment in C M. Am J Trop Med Hyg. 2008; 78:198-205.
9) Zimmerman G A, Castro-Faria-Neto H. Persistent cognitive impairment after CM: models, mechanisms and adjunctive therapies. Expert Rev Anti Infect Ther. 2010; 8:1209-12.
10) Gowda D C, Wu X. Parasite Recognition and Signaling Mechanisms in Innate Immune Responses to Malaria. Front Immunol. 2018; 19; 9:3006.
11) Olivier M, Van Den Ham K, Shio M T, Kassa F A, Fougeray S. Malarial pigment hemozoin and the innate inflammatory response. Front Immunol. 2014; 5:25.
12) Pamplona A, Ferreira A, Balla J, Jeney V, Balla G, Epiphanio S, Chora A, Rodrigues C D, Gregoire I P, Cunha-Rodrigues M, Portugal S, Soares M P, Mota M M. Heme oxygenase-1 and carbon monoxide suppress the pathogenesis of experimental C M. Nat Med. 2007; 13(6):703-710.
13) Ferreira A, Balla J, Jeney V, Balla G, Soares M P. A central role for free heme in the pathogenesis of severe malaria: the missing link? J Mol Med (Berl). 2008; 86:1097-111.
14) Ferreira A, Marguti I, Bechmann I, Jeney V, Chora A, Palha N R, Rebelo S, Henri A, Beuzard Y, Soares M P. Sickle hemoglobin confers tolerance to Plasmodium infection. Cell. 2011; 145:398-409.
15) Amodu O K, Adeyemo A A, Olumese P E, Gbadegesin R A. Intraleucocytic malaria pigment and clinical severity of malaria in children. Trans. R. Soc. Trop. Med. Hyg. 1998; 92: 54-56.
16) Newton C R, Taylor T E, Whitten R O. Pathophysiology of fatal falciparum malaria in African children. Am. J. Trop. Med. Hyg. 1998; 58:673-683.
17) Birhanu M, Birhanu M, Asres Y, Adissu W, Yemane T, Zemene E, Gedefaw L. Hematological parameters and hemozoin-containing leukocytes and their association with disease severity among malaria infected children: A cross-sectional study at Pawe general hospital, Northwest Ethiopia. Interdiscip. Perspect. Infect. Dis. 2017; 8965729.
18) Dalko E, Das B, Herbert F, Fesel C, Pathak S, Tripathy R, Cazenave P-A, Ravindran B, Sharma S, Pied S. Multifaceted role of heme during severe Plasmodium falciparum infections in India. Infect. Immun. 2015; 83: 3793-3799.
19) Elphinstone R E, Riley F, Lin T, Higgins S, Dhabangi A, Musoke C, Cserti-Gazdewich C, Regan R F, Warren H S, Kain K C. Dysregulation of the haem-haemopexin axis is associated with severe malaria in a case-control study of Ugandan children. Malaria J. 2015; 14:511.
20) de Souza J B, Hafalla J C, Riley E M, Couper K N. C M: why experimental murine models are required to understand the pathogenesis of disease. Parasitology 2010; 137:755-772.
21) Ghazanfari N, Mueller S N, Heath W R. Cerebral malaria in mouse and man. Front. Immunol. 2018; 9:2016.
22) Dorovini-Zis K, Schmidt K, Huynh H, Fu W, Whitten R O, Milner D, Kamiza S, Molyneux M, Taylor T E. The neuropathology of fatal C M in Malawian children. Am. J. Pathol. 2011; 178:2146-2158.
23) Franke-Fayard B, Fonager J, Braks A, Khan S M, Janse C J. Sequestration and tissue accumulation of human malaria parasites: can we learn anything from rodent models of malaria? PLoS Pathog. 2010; 6:e1001032.
24) Baptista F G, Pamplona A, Pena A C, Mota M M, Pied S, Vigirio A M. Accumulation of Plasmodium berghei-infected red blood cells in the brain is crucial for the development of C M in mice. Infect Immun. 2010; 78:4033-4039.
25) Strangward P, Haley M J, Shaw T N, Schwartz J M, Greig R, Mironov A, de Souza J B, Cruickshank S M, Craig A G, Milner Jr D A, Allan S M, Couper K N. A quantitative brain map of experimental C M pathology. PLoS Pathog. 2017; 13:e1006267.
26) Surolia N, Padmanaban G. De novo biosynthesis of heme offers a new chemotherapeutic target in the human malarial parasite. Biochem Biophys Res Commun. 1992; 187:744-750.
27) Padmanaban G, Nagaraj V A, Rangarajan, P N. (2013). Unique Features of heme biosynthesis in the malaria parasite. Handbook of Porphyrin Science. With Applications to Chemistry, Physics, Materials Science, Engineering, Biology and Medicine. World Scientific Publishers, Edited by Kadish, K. M., Smith, K. M., Guilard, R. and Ferreira, G. C. 27, 168-205.
28) Nagaraj V A, Padmanaban G. Insights on Heme Synthesis in the Malaria Parasite. Trends Parasitol. 2017; 33:583-586.
29) Nagaraj V A, Sundaram B, Varadarajan N M, Subramani P A, Kalappa D M, Ghosh S K, Padmanaban G. Malaria parasite-synthesized heme is essential in the mosquito and liver stages and complements host heme in the blood stages of infection. PLoS Pathog. 2013; 9:e1003522.
30) Chandana M, Anand A, Ghosh S, Beura S, Jena S, Suryawanshi A R, Padmanaban G, Nagaraj V A. Malaria parasite biosynthetic heme induces cerebral pathogenesis by regulating hemozoin formation and griseofulvin prevents cerebral malaria. (2021). bioRxiv. doi: https://doi.org/10.1101/2021.04.27.441715
31) WHO Model Prescribing Information: Drugs Used in Skin Diseases. World Health Organization. (1997). https://apps.who.int/iris/bitstream/handle/10665/41975/9241401060.pdf?sequence=1&isAllowed=y
32) Petersen A B, Ronnest M H, Larsen T O, Clausen M H. The chemistry of griseofulvin. Chem Rev. 2014; 114:12088-12107.
33) Liu K, Yan J, Sachar M, Zhang X, Guan M, Xie W, Ma X. A metabolomic perspective of griseofulvin-induced liver injury in mice. Biochem Pharmacol. 2015; 98: 493-501.
34) Smith C M, Jerkovic, A, Truong T T, Foote S J, McCarthy J S, McMorran B J. Griseofulvin impairs intraerythrocytic growth of Plasmodium falciparum through ferrochelatase inhibition but lacks activity in an experimental human infection study. Sci. Rep. 2017; 7: 41975.
35) WO1996016664—Compositions for the treatment of parasitic and fungal infections.
36) Ramakrishnan G, Chandra N, Srinivasan N. Exploring anti-malarial potential of FDA approved drugs: an in-silico approach. Malaria J. 2017; 16: 290.

### OBJECTS OF THE INVENTION

An object of this invention is to establish that the heme synthesized in malaria parasite is associated with cerebral malaria pathogenesis and disease severity.

Another object of this invention is to propose griseofulvin for preventing and treating cerebral and severe malaria.

Yet another object of the invention is to propose griseofulvin as an adjunct therapy. Still another object of this invention is to propose griseofulvin in standard dose along with standard ACTs used in malaria treatment.

Further object of the invention is to establish synergy of griseofulvin with α,β-arteether, the principal artemisinin component of ACTs and the frontline antimalarial, in terms of protection against cerebral malaria.

### SUMMARY OF INVENTION

Heme serves as a cofactor for cytochromes that act as electron carriers in the electron transport chain (ETC). The asexual stages possess a primitive mitochondrion that lacks cristae and depend primarily on glycolysis for ATP generation. The major function of ETC in the asexual stages is to support pyrimidine biosynthesis and maintain mitochondrion membrane potential. Heme pathway is dispensable for asexual stages and the parasite has evolved with backup mechanisms to acquire Hb-heme, porphyrin intermediates and host enzymes. The question arises regarding the expression of heme pathway enzymes and the ability of asexual parasites to synthesize heme despite having backup mechanisms. Here, it has been demonstrated that de novo heme pathway is associated with disease virulence and it promotes CM pathogenesis by enhancing Hz formation. Mice infected with heme pathway knockout (KO) parasites synthesize less hemozoin and are devoid of cerebral pathogenesis. The decrease in Hz synthesis is reflected in the reduced Hz load in spleen and liver, and decreased free heme levels in the KO parasites and plasma samples of KO-infected mice. Hz is a malarial PAMP that can be directly associated with aberrant inflammatory responses and cerebral pathogenesis. Mice infected with KO parasites are devoid of cerebral complications. The plasma levels of IL-6, TNFα, IFNγ, G-CSF, CCL3 and CCL5 are significantly decreased in KO-infected mice. The decrease in pro-inflammatory cytokines and chemokines together with the concomitant increase in anti-inflammatory cytokines such as IL-4, IL-10 and IL-13 in the KO-infected mice indicate an over-all decrease in the systemic inflammation.

The transcript levels of cytokines and chemokines such as TNFα, IFNγ, CXCL9, CXCL10, CCL2, CCL5 and CCL19 are low in the brain samples of KO-infected mice. The brain of KO-infected mice shows reduction in infiltration of CD8⁺ T cells that express perforin, granzyme B, IFNγ, TNFα and CXCR3. These effector molecules can enhance CD8⁺ T cell migration, cytotoxicity and cross presentation of antigens by endothelial cells, promote pRBC sequestration, and induce apoptosis in neurons, endothelial cells and astrocytes. Consistent with these findings, a decrease in phospho-NF-κB—the transcription factor that upregulates NLRP3 inflammasome is observed in the brain homogenates of KO-infected mice. There is also a reduction in the NLRP3 inflammasome formation as evident from decreased levels of phsopho-NLRP3 leading to decreased caspase-1 activation and less production of IL-1β—a key mediator of inflammation, infiltration of immune cells and apoptosis. The fatality rates in malaria do not correlate with parasite clearance and therefore, targeting parasite virulence is also important. The present invention showed that griseofulvin prevents CM in mice by inhibiting parasite heme synthesis and delays death due to anemia. By the observation of the inventors, griseofulvin can serve as an excellent synergistic adjunct to ACTs and other frontline antimalarial therapies for CM and other forms of severe malaria. The present invention also claims that griseofulvin combination with the existing ACTs or other ACTs in the process of development or other antimalarials has a direct industrial application for developing a new adjunct therapy.

Therefore, in an embodiment, the present invention discloses and claims a pharmaceutical composition against cerebral and severe malaria comprising artemisinin-based combination therapy (ACT), in combination with griseofulvin, for three days, wherein 500-1000 mg/day of griseofulvin is used in adults and 5-10 mg/kg/day of griseofulvin in children, in combination with ACTs, characterized in that, the ACTs that are recommended are α,β-arteether with lumefantrine or artemether in combination with lumefantrine or artesunate in combination with amodiaquine or artesunate in combination with mefloquine or dihydroartemisinin in combination with piperaquine or artesunate in combination with sulfadoxine-pyrimethamine.

In another embodiment, the present invention discloses a method of preventing and treating cerebral and severe malaria comprising administering of griseofulvin in combination with artemisinin-based combination therapy (ACT), for three days, wherein 500-1000 mg/day of griseofulvin is used in adults and 5-10 mg/kg/day of griseofulvin in children, in combination with ACTs, wherein the ACTs that are recommended are α,β-arteether with lumefantrine or artemether in combination with lumefantrine or artesunate in combination with amodiaquine or artesunate in combination with mefloquine or dihydroartemisinin in combination with piperaquine or artesunate in combination with sulfadoxine-pyrimethamine.

In another embodiment, the present invention discloses griseofulvin for use in a method of prevention or treatment of cerebral or severe malaria, in combination with artemisinin-based combination therapy (ACT), wherein 500-1000 mg/day of griseofulvin is used in adults and 5-10 mg/kg/day of griseofulvin in children in combination with ACTs, and wherein the ACTs that are recommended are α,β-arteether with lumefantrine or artemether in combination with lumefantrine or artesunate in combination with amodiaquine or artesunate in combination with mefloquine or dihydroartemisinin in combination with piperaquine or artesunate in combination with sulfadoxine-pyrimethamine.

### BRIEF DESCRIPTION OF ACCOMPANYING FIGURES/DRAWINGS

FIG. 1: Characterization of heme pathway KO parasites in C57BL/6 mice. A) Growth curve analysis of PbWT (n=10), PbALASKO (n=12) and PbFCKO (n=12) parasites in C57BL/6 mice. 10⁵ parasites were used to initiate WT and KO parasite infections. The data represent three different batches. B) Mortality curves of mice infected with PbWT, PbALASKO and PbFCKO parasites. The data represent the mice utilized for growth curve analysis. C) Spleen weight of mice infected with PbWT (n=13), PbALASKO (n=14) and PbFCKO (n=14) parasites. For each day, 3-5 mice from four different batches were included. D) Growth curve analysis of PbWT (n=14), PbALASKO (n=14) and PbFCKO (n=14) parasites in C57BL/6 mice. 10⁵ and 10⁷ parasites were used to initiate WT and KO parasite infections, respectively. The data represent four different batches. E) Mortality curves of mice infected with PbWT, PbALASKO and PbFCKO parasites. The data represent the mice utilized for growth curve analysis. F) Percentage of infected reticulocytes in the parasitized red cells. The data represent six mice each for WT, ALASKO and FCKO parasites. G) Giemsa stained images for peripheral blood smears prepared from tail vein blood of WT and KO parasite-infected mice. Images were captured using 100× objective lens. Scale bar=5 μm. H) RMCBS score for mice infected with WT (n=8) and KO (n=12) parasites. ***P<0.001.

FIG. 2: In vivo bioluminescence imaging of C57BL/6 mice infected with heme pathway KO parasites. A) Double crossover recombination strategy utilized to generate Luc-expressing ALASKO and FCKO parasites. B) PCR confirmation for ALAS and FC deletions with genomic DNA isolated from Luc-expressing ALASKO and FCKO parasites. Lane M: 1 kb ladder; Lane 1, 3 and 5: ALAS product (2.11 kb); Lane 2, 4 and 6: FC product (1.54 kb). C) RT-PCR confirmation for ALAS and FC deletions with total RNA isolated from Luc-expressing ALASKO and FCKO parasites. Lane M: 1 kb ladder; Lane 1, 3 and 5: ALAS product (1.92 kb); Lane 2, 4 and 6: FC product (1.05 kb). D) Live GFP and m-cherry fluorescence of Luc-expressing WT and heme pathway KO parasites. Images were captured using 100× objective lens. Scale bar=5 μm. E) Whole body bioluminescence imaging of WT and KO parasite-infected mice on day 8 post-infection. F) Ex vivo bioluminescence imaging of liver (Li), lungs (Lu), brain (B), heart (H) and spleen (S) of WT and KO parasite-infected mice. Enlarged images of brain are presented below. Luminescence scale represents radiance (p/sec/cm²/sr). G) Mortality curves of mice infected with Luc-expressing PbWT (n=5), PbALASKO (n=4) and PbFCKO (n=5) parasites. ***P<0.001.

FIG. 3: Assessment of cerebral pathology in C57BL/6 mice infected with heme pathway KO parasites. A) Extravasation of Evans blue in the brain of mice infected with WT and heme pathway KO parasites. B) Quantification of Evans blue in the brain samples of mice infected with WT (n=6) and heme pathway KO (n=9) parasites. C) H&E staining of the brain sections prepared from WT and KO parasite-infected mice. Black arrows indicate intracerebral and petechial hemorrhages, blue arrows indicate thrombosed blood vessels and brown arrows indicate gross demyelination. Images were captured using 10× objective lens. Scale bar=50 μm. D) Immunohistochemical analysis of IgG extravasation in the brain sections of WT and KO parasite-infected mice. Black arrows indicate the areas showing IgG immunoreactivity. Images were captured using 10× objective lens. Scale bar=50 μm. E) H&E staining of the brain sections prepared from WT and KO parasite-infected mice. Black arrows indicate occluded vasculatures containing luminal and abluminal leukocytes, and parasite-derived hemozoin. Images were captured using 60× objective lens. Scale bar=10 μm. F) Immunofluorescence analysis of parasite accumulation in the brain sections of WT and KO parasite-infected mice. G) Immunofluorescence analysis of CD3⁺ cells in the blood vessels of WT and KO parasite-infected mice. H) Immunofluorescence analysis of β-APP staining in the brain sections of WT and KO parasite-infected mice. Images were captured using 20× objective lens. Scale bar=20 μm. ***P<0.001.

FIG. 4: Assessment of inflammatory parameters in WT and heme pathway KO parasites. A) Cytokine and chemokine levels in the plasma samples of WT-, ALASKO and FCKO-infected mice (n=5). B) qPCR analyses of host transcripts in the brain samples of infected mice. Expression levels were normalized with GAPDH. Relative expression fold changes of mRNA transcripts in the KO-infected mice with respect to WT-infected mice are shown (n=3). C) Flow cytometry analyses of T cells in the brain samples of infected mice. Mice on day 7/8 post-infection were used and the data for each cell type were obtained from three different mice infected with WT or KO parasites. D) Western analyses of brain homogenates prepared from WT- and KO-infected mice. 200 μg of total protein was used from the pooled brain homogenates of three different mice. *P<0.05; **P<0.01; ***P<0.001.

FIG. 5: Hemozoin and free heme levels in WT and heme pathway KO parasites. A) Bright field images of Giemsa stained WT, ALASKO and FCKO asexual stage parasites in peripheral blood smears showing hemozoin content. B) Giemsa stained images of WT, ALASKO and FCKO gametocytes. C) Hemozoin content in differential interference contrast (DIC) and bright field images of paraformaldehyde-fixed RBCs containing WT and KO parasites. Images were captured using 100× objective lens. Scale bar=5 μm. D) Hemozoin levels in WT and KO parasites. E) Free heme levels in WT and KO parasites. F) Free heme levels in the plasma samples of WT and KO parasite-infected mice. G) Heme/Hemopexin ratio in the plasma samples of WT and KO parasite-infected mice. H) Plasma hemopexin levels of WT and KO parasite-infected mice. I) Plasma hemoglobin levels of WT and KO parasite-infected mice. J) Hemozoin load in the spleen of WT and KO parasite-infected mice. K) Hemozoin load in the liver of WT and KO parasite-infected mice. The data represent nine mice each for WT, ALASKO and FCKO. *P<0.05; **P<0.01; ***P<0.001.

FIG. 6: Effect of griseofulvin treatment on cerebral malaria pathogenesis. A) Mortality curves of PbWT-infected mice treated with different dosages of griseofulvin. The control mice were injected with solvent (1 or 2 doses) on day 4, 5, 6, 7 and 8. B) Growth curve analysis of griseofulvin-untreated (n=12) and -treated (n=12) PbWT parasites in C57BL/6 mice. The griseofulvin treated data represent 2 mg dose per day on day 4, 5, 6, 7 and 8. C) Phosphorimager and scanned images of TLC performed for ¹⁴C-ALA labelled free heme isolated from griseofulvin-untreated and -treated PbWT parasites and the radioactive counts measured. The radioactive counts represent the data from three experiments. D) Extravasation of Evans blue and its quantification in the brain samples of girseofulvin-treated mice infected with WT parasites. The brain samples were isolated on day 8 post-infection (n=3). E) H&E staining of the brain sections prepared from griseofulvin-treated WT parasite-infected mice. Images were captured using 10× objective lens. Scale bar=50 μm. F-H) Immunofluorescence analysis of parasite accumulation, CD3⁺ cells in the blood vessels and axonal injury in the brain sections of griseofulvin-treated WT parasite-infected mice. Images were captured using 20× objective lens. Scale bar=20 μm. I) Bright field images of Giemsa stained griseofulvin-treated WT parasite in peripheral blood smears showing hemozoin content. J) Hemozoin content in differential interference contrast (DIC) and bright field images of paraformaldehyde-fixed RBCs containing griseofulvin-treated WT parasites. Images were captured using 100× objective lens. Scale bar=5 μm. K) Hemozoin levels in griseofulvin-treated WT parasites (n=5). L) Free heme levels in griseofulvin-treated WT parasites (n=5). M) Free heme levels in the plasma samples of griseofulvin-treated WT parasite-infected mice (n=6). N) Plasma hemopexin levels of griseofulvin-treated WT parasite-infected mice (n=6). O) Heme/Hemopexin ratio in the plasma samples of griseofulvin-treated WT parasite-infected mice. P) Plasma hemoglobin levels of griseofulvin-treated WT parasite-infected mice (n=11). *P<0.05; **P<0.01; ***P<0.001.

FIG. 7: Synergy of griseofulvin with α,β-arteether in preventing CM in mice. A) Dose standardization for α,β-arteether treatment leading to CM in mice. The respective doses were given intramuscularly on day 6 when the parasitemia was around 10%. The data represent five mice from each group. B) Synergistic combination of α,β-arteether and griseofulvin preventing CM in mice. The mice were treated with a single dose of 0.25 mg α,β-arteether on day 6, followed by two doses of griseofuivin (2 mg per day) on day 6 and 7, post α,β-arteether treatment. For control, mice were treated with a single dose of 0.25 mg α,β-arteether on day 6. The data represent nine mice from each group. C) Growth curve analysis of mice treated with α,β-arteether alone and α,β-arteether in combination with griseofulvin. The data represent the mice utilized for mortality analyses. ***P<0.001.

### DETAILED DESCRIPTION OF THE INVENTION

At the very outset of the detailed description, it may be understood that the ensuing description only illustrates a form of this invention. However, such a form is only exemplary embodiment, and without intending to imply any limitation on the scope of this invention. Accordingly, the description is to be understood as an exemplary embodiment and teaching of invention and not intended to be taken restrictively.

Throughout the description and claims of this specification, the phrases “comprise” and “contain” and variations of them mean “including but not limited to”, and are not intended to exclude other moieties, additives, components, integers or steps. Thus, the singular encompasses the plural unless the context otherwise requires. Wherever there is an indefinite article used, the specification is to be understood as contemplating plurality as well as singularity, unless the context requires otherwise.

Thus, the terms “comprise”, “comprising”, or any other variations thereof used in the disclosure, are intended to cover a non-exclusive inclusion, such that a device, system, assembly that comprises a list of components does not include—only those components but may include other components not expressly listed or inherent to such system, or assembly, or device.

In other words, one or more elements in a system or device proceeded by “comprises . . . a” does not, without more constraints, preclude the existence of other elements or additional elements in the system, apparatus or device.

Features, integers, characteristics, compounds, chemical moieties or groups described in conjunction with an aspect, embodiment or example of the invention are to be understood to be applicable to any other aspect, embodiment or example described herein unless incompatible therewith. All the features disclosed in this specification (including any accompanying claims, abstract and drawings), and/or all of the steps of any method or process so disclosed, may be combined in any combination, except combinations where at least some of such features and/or steps are mutually exclusive.

The invention is not restricted to the details of any foregoing embodiments. The invention extends to any novel one, or any novel combination, of the features disclosed in this specification including any accompanying claims, abstract and drawings or any parts thereof, or to any novel one, or any novel combination, of the steps of any method or process so disclosed.

The reader's attention is directed to all papers and documents which are filed concurrently with or before this specification in connection with this application and which are open to public inspection with this specification, and the contents of all such papers and documents are incorporated herein by reference. Post filing patents, original peer reviewed research paper shall be published.

The following descriptions of embodiments and examples are offered by way of illustration and not by way of limitation.

Unless contraindicated or noted otherwise, throughout this specification, the terms “a” and “an” mean one or more, and the term “or” means and/or. As used in the description herein and throughout the claims that follow, the meaning of “a.” “an,” and “the” includes plural reference unless the context clearly dictates otherwise. Also, as used in the description herein, the meaning of “in” includes “in” and “on” unless the context clearly dictates otherwise.

Further, it may also be noted that throughout the specification, some spelling of words may be put in American English and also in British English, for example “Fibre” may also be written as “Fiber”, and the likes. No confusion may be there regarding the actual meaning of any such word, differed only in spelling owing to different practices.

Mortality in cerebral malaria (CM) and severe malaria remains high, despite parenteral administration of artemisinin derivatives or quinine with supportive symptomatic therapies. The fatality rates do not correlate with parasite clearance and targeting parasite virulence becomes important. The present studies carried out in C57BL/6 mice using the rodent parasite P. berghei ANKA, a well-established in vivo animal model to study human CM have shown that de novo heme synthesis of malaria parasite is required to develop CM pathogenesis by enhancing hemozoin (Hz) formation. Mice infected with heme pathway knockout (KO) parasites are devoid of cerebral complications and are completely protected from CM. The death due to anemia that happens in the absence of any therapeutic intervention is also delayed in the mice infected with KO parasites. Mice infected with KO parasites show an overall decrease in systemic and neuronal inflammation, and T cell infiltration in the brain milieu. Therefore, inhibitors of the parasite heme-biosynthetic pathway have the potential to prevent cerebral and severe malaria. The technical advance in this invention is that griseofulvin, an FDA-approved antifungal drug, can inhibit parasite heme synthesis and prevent onset of cerebral pathology, although parasite growth is not inhibited. Thus, griseofulvin exhibits synergy with artemisinin derivative—α,β-arteether in terms of protecting mice from cerebral malaria. Therefore, a combination of artemisinin-based combination therapies or other antimalarials used as frontline therapies for malaria to inhibit parasite growth, together with griseofulvin as an adjunct drug would serve as a new product and therapeutic combination for preventing cerebral malaria and severe malaria in humans.

The present invention explored the possibility of including griseofulvin in the treatment protocol in affected patient population. The only inclusion will be griseofulvin for three days to prevent the development of cerebral and severe malaria. In humans, griseofulvin dosage is given to the extent of 500-1000 mg/day in adults and 5-10 mg/kg/day in children for several weeks. The conversion of 5-10 mg/kg/day into animal dose is carried out by multiplying human dose with 12.3. Taking the average weight of 7-8 weeks mouse as 25 g, this comes to 1.5-3.0 mg per mouse. Therefore, the dose used in this invention for griseofulvin (2 mg per mouse per day) is matching to human dose that has been routinely used for the treatment of fungal infections.

It may especially be noted that griseofulvin is not an antimalarial drug, and has no known direct effect against the parasitic growth. However, the present invention suggests prevention of cerebral malaria and disease severity, and a consequent measure to avert mortality caused by malaria that occurs despite treating the patients with ACTs. Further, it must be noted that the existing WHO-approved treatment for malaria to be followed and no changes has been suggested in the dosage or treatment regimen of ACTs. Griseofulvin will be included in the treatment protocol for three days at the same dosage (500-1000 mg/day in adults and 5-10 mg/kg/day for children) that has been used for fungal diseases along with the existing ACTs to prevent cerebral and severe malaria. The treatment protocol suggested in this invention will be the standard ACT that has been given as per the WHO recommendation for malaria patients along with griseofulvin for three days starting from the first day of ACT treatment. There will not be any change in the standard malaria therapy except that the griseofulvin will be included for three days. The following are the ACTs recommended by WHO: 1) artemether/arteether+lumefantrine 2) artesunate+amodiaquine 3) artesunate+mefloquine 4) dihydroartemisinin+piperaquine 5) artesunate+sulfadoxine-pyrimethamine for malaria. Any one of these will be used in malaria endemic regions based on national policies, resistance pattern in that particular region etc. This ACT treatment is given for 3 days.

Mice Infected with Heme Pathway KO Parasites are Protected from CM

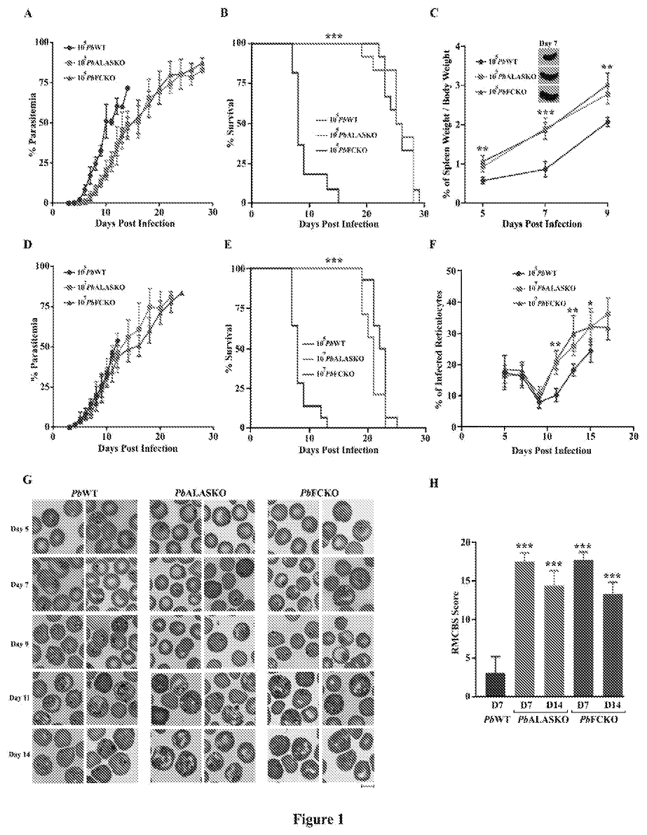

Growth analyses for the P. berghei KO parasites in CM-susceptible C57BL/6 inbred mouse strain was carried out by injecting 10⁵ asexual stage parasites. Assessment of the peripheral blood parasitemia showed 2-3 days delay in the growth of KO parasites with respect to the wild-type (WT) parasites. Importantly, about 80% of the WT-infected mice succumbed to CM within day 10 when the blood parasitemia was around 10-30% showing typical symptoms of CM. The WT-infected mice that could escape from CM died of anemia on day 12-16 post-infection. In contrast, all the mice infected with ALASKO and FCKO parasites were protected from CM and they died because of anemia on day 20-30. The delay in the growth of KO parasites was associated with an early increase in the spleen weight of the infected mice suggesting a better splenic clearance. To rule out the possibility of protection from CM is because of a delay in the blood parasitemia increase, growth analyses in C57BL/6 mice infected with 10⁷ ALASKO and FCKO parasites were performed. While the growth of KO parasites in mice injected with 10⁷ parasites was comparable with 10⁵ WT parasites, mice infected with KO parasites were once again completely protected from CM. The mortality of the KO-infected mice due to anemia was also delayed by almost 6-10 days, and the KO-infected mice could sustain a higher blood parasitemia for a prolonged period. There were no significant differences in the reticulocyte versus mature RBC preference between WT and KO parasites in the first 9 days of infection, the duration in which the CM mortality occurred in WT-infected mice. However, in comparison with WT, KO parasites showed significantly increased reticulocyte preference and multiple infections in the reticulocytes during the later course of infections that represent the anaemic phase. The rapid murine coma and behavioural scale (RMCBS) score of 10⁵ WT parasite-infected mice that succumbed to CM was below 5 on day 7 whereas, RMCBS score of 10⁷ KO parasite-infected mice was around 17 and 14 on day 7 and 14, respectively (FIG. 1).

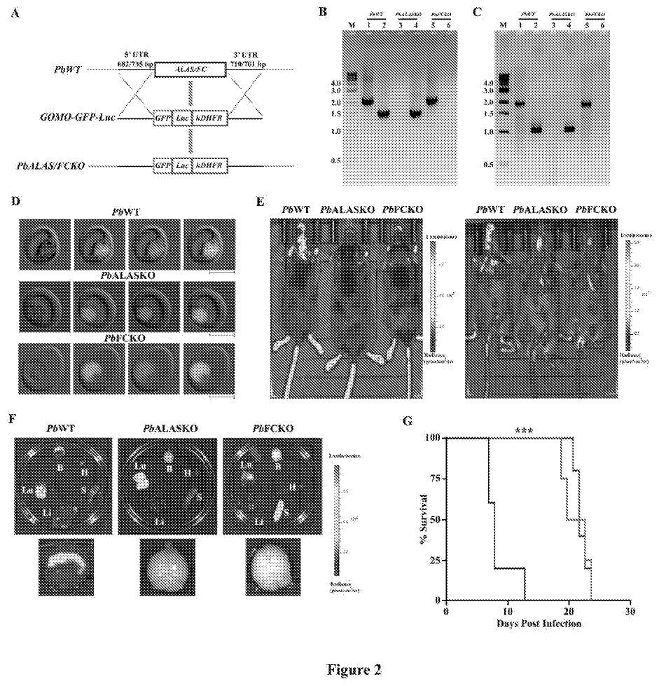

These results were confirmed with another set of independent KO parasite lines wherein, ALAS and FC genes were replaced individually with GFP-luciferase (Luc)-expressing cassette containing m-cherry. The successful replacement of ALAS and FC was confirmed by PCR analyses performed with DNA and RNA isolated from the respective KO parasites, and by examining GFP and m-cherry fluorescence. For control, c/d ssurRNA locus in the WT parasite was replaced with GFP-Luc-expressing cassette. In vivo bioluminescence studies carried out for mice infected with Luc-expressing WT and KO parasites showed the accumulation of pRBCs in the brain of WT-infected mice, but not in the KO-infected mice. This was confirmed by performing ex vivo imaging for the perfused brain collected from WT- and KO-infected mice. The mice infected with Luc-expressing KO parasites were also completely protected from CM (FIG. 2).

Absence of Cerebral Pathology in Heme Pathway KO-Infected Mice

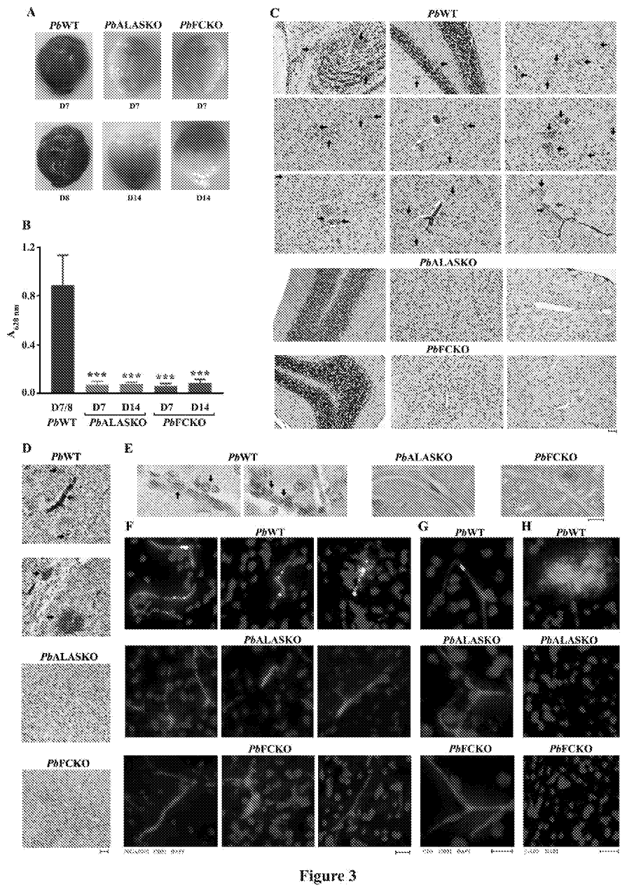

To evaluate the integrity of BBB and assess vascular leakage, Evans blue extravasation analyses were carried out. While the brain collected from WT-infected mice on day 7/8 stained intensely with the intravenously injected Evans blue, the extravasation of Evans blue into the brain was barely detectable in KO-infected mice on day 7 and 14. Quantification of Evans blue in the brain extracts confirmed this observation. Histopathological assessment of hematoxylin and eosin (H&E) stained brain sections of WT-infected mice on day 7 showed intracerebral hemorrhages with extravasation of erythrocytes into the perivascular space, petechial hemorrhages, thrombosed and leukocyte-packed vessels, gross demyelination and myelin pallor. No such hallmark features of CM could be detected in the brain sections of KO-infected mice. Immunohistochemical studies carried out with the brain sections of WT-infected mice showed the extravasation of IgG in cerebral parenchyma and the presence of IgG in occluded vessels and hemorrhages, but not in the KO-infected mice. The luminal and abluminal leukocytes, and parasite-derived Hz could also be detected in the occluded vasculature of WT-infected mice. Immunofluorescence analyses of the brain sections using Pb glyceraldehyde-3-phosphate dehydrogenase (GAPDH) and mouse CD31 antibodies showed the accumulation of parasites in CD31⁺ vasculature and extravascular parasites in the hemorrhages of WT-infected mice, but not in the KO-infected mice. Similarly, antibodies specific for CD3 and β-amyloid precursor protein (β-APP) showed the accumulation of CD3⁺ T cells in the cerebral vasculature and axonal injury in the brain sections of WT-infected mice, but not in the KO-infected mice. All these data indicated that the CM-associated brain lesions are absent in the mice infected with heme pathway KO parasites (FIG. 3).

Decreased Levels of Inflammatory Parameters in Heme Pathway KO-Infected Mice

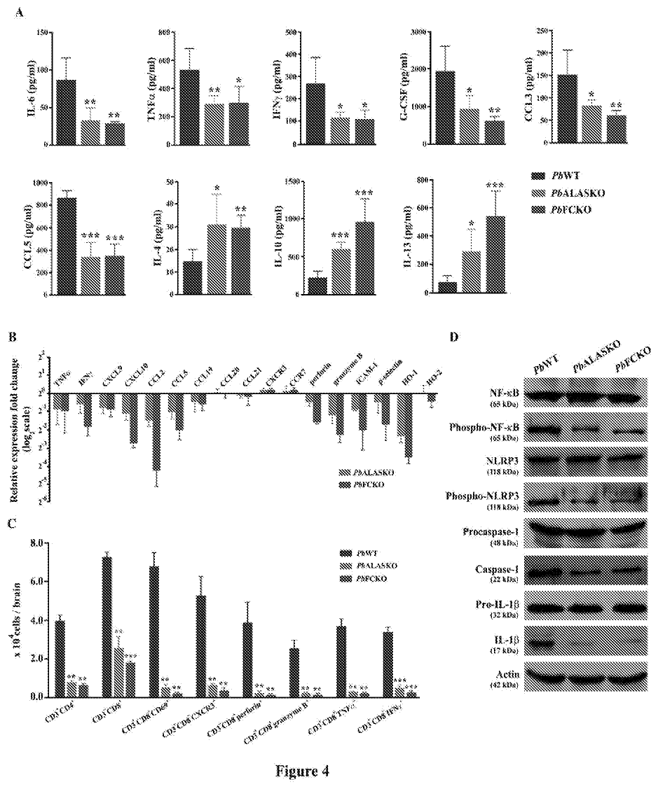

Multiplex assays carried out for plasma cytokines and chemokines in mice infected with KO parasites on day 8 showed a significant decrease in the levels of IL-6, TNFα, IFNγ, G-CSF, CCL3 (MIP-1α) and CCL5 (RANTES). There was also a significant increase in the levels of anti-inflammatory cytokines—IL-4, IL-10 and IL-13. Quantitative RT-PCR analyses carried out to examine the expression of cytokines, chemokines, chemokine receptors and other key mediators of cerebral pathogenesis in the brain samples of KO-infected mice showed a substantial reduction of more than 1.5-fold in the transcript levels of TNFα, IFNγ, CXCL9, CXCL10, CCL2 (MCP-1), CCL5, CCL19, perforin, granzyme B, ICAM-1, p-selectin and HO-1. In particular, the decrease in the expression levels of IFNγ, CXCL10, CCL2, CCL5, granzyme B, ICAM-1 and HO-1 in FCKO was greater than 4-fold and the observed decrease was more in case of FCKO than ALASKO. It is known that the infiltration of CD8⁺ T cells expressing early activation marker CD69, and proinflammatory and cytotoxic effector molecules such as TNFα⁺, IFNγ⁺, CXCR3⁺, perforin and granzyme B leads to endothelial leakage, BBB disruption and neuronal damage in CM. Flow cytometry analyses of the leukocytes isolated form the perfused brain samples of KO-infected mice showed significant reduction in CD3⁺ CD4⁺ and CD3⁺ CD8⁺ double positive cells, and CD3⁺ CD8⁺ CD69⁺, CD3⁺ CD8⁺ CXCR3⁺, CD3⁺ CD8⁺ perforin*, CD3⁺ CD8⁺ granzyme B⁺, CD3⁺ CD8⁺ TNFα⁺ and CD3⁺ CD8⁺ IFNγ⁺ triple positive cells. These data indicate an overall decrease in systemic inflammation, inflammation-associated pathogenesis and T cell infiltration in the brain milieu of KO-infected mice. Western analyses for the brain homogenates of KO-infected mice showed reduction in phospho-NLRP3, phospho-NF-κB, cleaved caspase-1 and IL-1β that are key mediators of neuroinflammation, cerebrovascular dysfunction, endothelial activation, and endothelial and neuronal apoptosis. All these evidences suggested that the cerebral pathology is impeded in mice infected with heme pathway KO parasites (FIG. 4).

Decreased Hz Formation in the KO Parasites

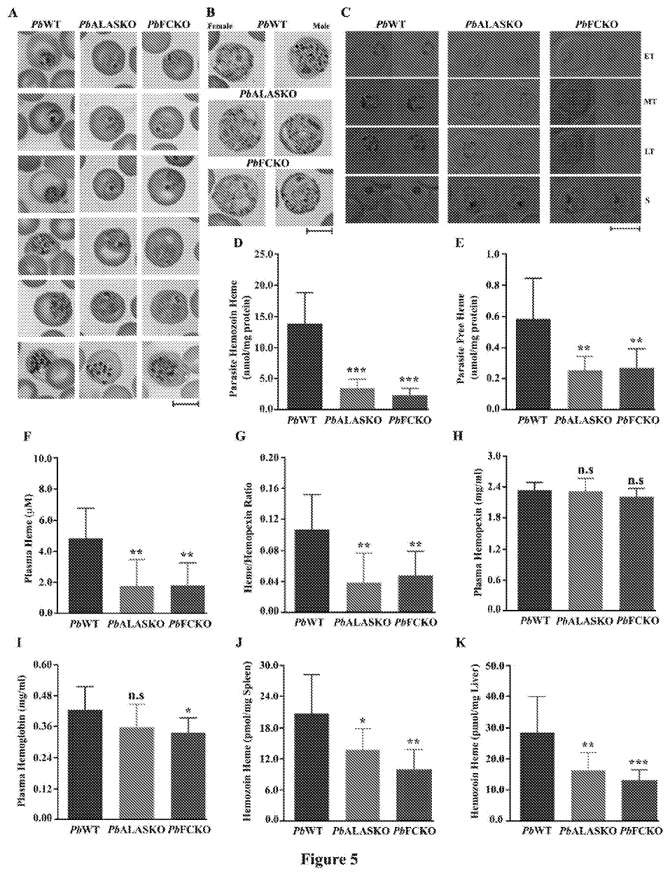

Light microscopy analyses of the Giemsa stained peripheral blood smears indicated a substantial decrease with Hz formation in the asexual stages (trophozoites and schizonts) of KO parasites in comparison to WT parasites. The observed decrease in the Hz formation was more prominent in the asexual stages than gametocytes. Such decreased levels of Hz could be readily detected in almost 60-70% of the pRBCs containing asexual stages. These findings were verified by examining the Hz content in paraformaldehyde-fixed pRBCs. To further confirm these findings, total Hz content of the parasites normalized with respect to the protein was examined. The total Hz content of the KO parasites was only 20-25% of the WT. It is known that Hb degradation and Hz formation in the FV results in the leaching of heme into the parasite cytosol. Free heme in the parasite lysates of the KO parasites prepared by hypotonic lysis showed around 55% decrease when compared with WT. In addition, there was also a significant decrease of around 55-60% in the plasma free heme and heme/hemopexin ratio of the KO-infected mice. However, there were no significant differences between the plasma hemopexin levels of WT- and KO-infected mice, and there was only a marginal 15-20% decrease in the plasma Hb levels that turned out to be significant in case of FCKO, but not in ALASKO. The quantification of Hz load in the spleen and liver showed 40-50% decrease in the KO-infected mice. These results suggested an overall decrease in the Hz synthesis of KO parasites (FIG. 5).

Griseofulvin Treatment Protects Mice from CM

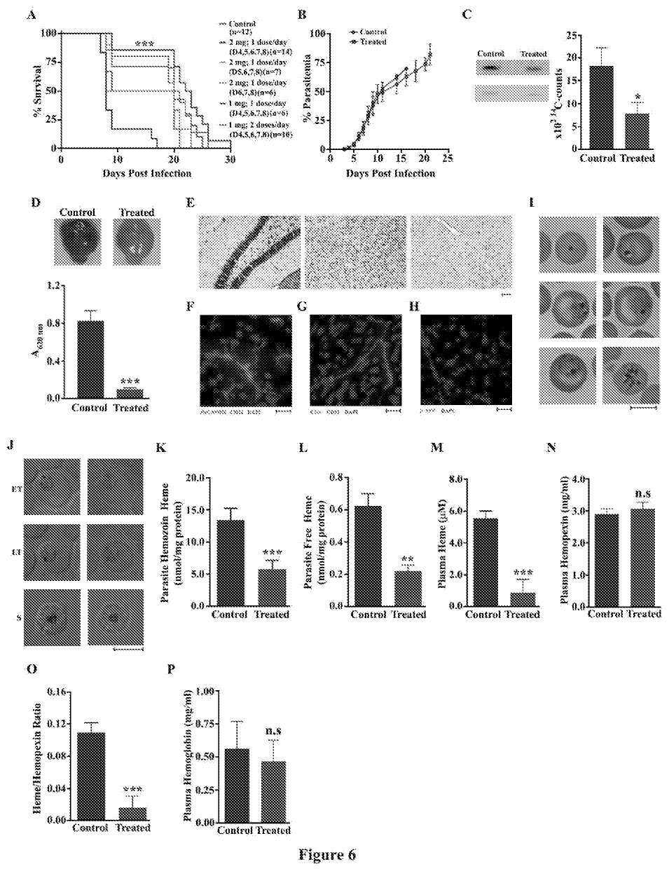

The present invention evaluated the potential of griseofulvin in preventing CM and treated WT-infected C57BL/6 mice from day 4 when the blood parasitemia was around 2%, and examined for CM pathogenesis. A single dose of 2 mg/day administered from day 4 and continued until day 8 showed the best protection from CM, a dosage that is very much comparable with the dosage of humans. While ˜80% of the control mice succumbed to CM within day 10, more than 80% of the griseofulvin-treated mice were protected from CM. Similar protection was also observed for the mice treated with 1 mg dose, twice a day from day 4 to day 8. In addition to CM protection, there was a significant delay in the mortality of the treated mice that occurred due to anemia. While the anemia mortality in CM-escaped control mice occurred within day 17, more than 80% of the griseofulvin-treated mice could survive beyond day 20 with almost 50% of them surviving even beyond day 24. Interestingly, the growth curves of WT parasites in treated and untreated mice were very much comparable, suggesting that CM protection is due to the inhibition of parasite heme synthesis, and not because of mitosis and parasite growth. This is also in agreement with the clinical trial performed in humans infected with Plasmodium falciparum: wherein, griseofulvin treatment did not inhibit the in vivo parasite growth (34). To confirm that the CM protection is due to inhibition of parasite heme synthesis, the present invention analyzed heme synthesis in griseofulvin-treated and -untreated WT parasites by incubating the in vivo-treated pRBCs in vitro with ¹⁴C-ALA (committed precursor of heme synthesis) for 9 h. There was around 60% decrease in the ¹⁴C-labelling of free heme in griseofulvin-treated parasites. Further, griseofulvin-treated mice showed less Evans blue extravasation in the brain, suggesting the preservation of BBB integrity. Histochemical and immunofluorescence analyses of the brain sections indicated the absence of intracerebral hemorrhages, and lack of accumulation of parasites and CD3⁺ T cells in the cerebral vasculature. This was also the case for axonal injury examined by β-APP staining. Light microscopy analyses of Giemsa-stained peripheral smears and paraformaldehyde-fixed pRBCs showed less Hz content. As observed for the KO parasites, the total Hz content in the griseofulvin-treated parasites was around 50-60% less when compared with untreated parasites and there was also close to 60% decrease in the free heme levels. Further, the plasma levels of heme, hemopexin and Hb together with heme/hemopexin ratio of the griseofulvin-treated WT-infected mice were found to be comparable to that of the KO-infected mice. Altogether, these results indicated the potential of griseofulvin in preventing CM and disease severity through the inhibition of parasite heme synthesis (FIG. 6).

Griseofulvin Exhibits Synergy with α,β-Arteether in Preventing CM in Mice

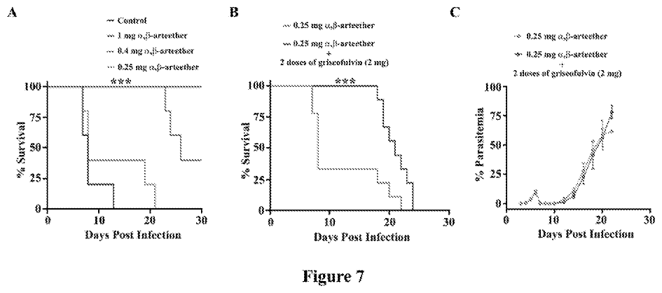

The present invention examined the potential of griseofulvin as an adjunct drug with α,β-arteether (primary artemisinin component of ACTs) in preventing CM in mice. The dose optimization studies performed with α,β-arteether indicated that a single intramuscular dose of 0.25 mg/mouse on day 6 when the parasitemia was around 10% resulted in CM in ˜60-70% of WT-infected mice, despite the clearance of peripheral parasitemia in blood smears. However, the inclusion of two doses of 2 mg griseofulvin at 24 h interval on day 6 and 7, post-α,β-arteether treatment, led to a complete protection of CM. The synergistic outcome of CM protection in mice treated with the combination of α,β-arteether and griseofulvin in comparison with α,β-arteether alone was observed with not much significant differences in day 7 parasite load and in the mortality due to anemia caused by recrudescence. While the two drugs act differently—α,β-arteether being responsible for clearing/reducing the parasite load and griseofulvin responsible for preventing cerebral pathogenesis, the combination is effective in bringing out a synergistic outcome in terms of preventing CM mortality. (FIG. 7).

Now, the invention will be described through illustrative examples. The examples given are merely illustrative of the uses, processes and products claimed in this invention, and the practice of the invention itself is not restricted to or by the examples described.

### Example 1

Routine propagation of P. berghei in mice and CM experiments. P. berghei ANKA WT and KO parasites were propagated in C57BL/6 male/female mice of 7-8 weeks old. Peripheral blood parasitemia was monitored by performing light microscopy for Giemsa stained thin blood smears prepared from tail vein blood. When the blood parasitemia was around 10%, 10⁵ P. berghei ANKA WT or 10⁵/10⁷ ALAS/FC KO parasites were collected and injected intraperitoneally in 7-8 weeks old C57BL/6 male/female naïve mice to initiate CM experiments. Growth curve analysis was carried out by monitoring the blood parasitemia. The development and progression of ECM were monitored by examining RMCBS for neurological symptoms. To assess BBB integrity, Evans blue uptake assays were carried out by injecting 200 μl of 2% Evans blue in PBS intravenously and examining the extravasation of dye after one hour in the brain of the infected mice that were transcardially perfused with PBS. The extent of BBB damage was quantified by incubating the brain samples in formamide at 37° C. for 48 h, extracting the Evans blue and measuring the absorbance at 620 nm.

### Example 2

Histological and immunofluorescence analyses of cerebral pathology in the brain of infected mice. For H&E staining to assess vascular blockage, hemorrhages and demyelination, brain samples were fixed with formalin for 72 hours at room temperature. After dehydrating with ethanol and treating with xylene, paraffin embedded blocks were made and sections of 7 μm thickness were prepared using rotary microtome. The sections were then processed and stained with H&E using standard protocols. Immunohistochemical analysis of IgG extravasation in the brain sections was carried out as described. Brain sections of 30 μm thickness was antigen retrieved by treating them at 95° C. for 30 min in sodium citrate buffer pH 6.0, followed by blocking with 3% H₂O₂ at room temperature for 30 min to prevent endogenous peroxidase activity. The sections were then incubated with HRP-conjugated goat anti-mouse IgG at 1:250 dilution in PBS containing 0.3% Triton X-100 and 0.1% BSA, followed by developing with diaminobenzidine tetrahydrochloride and counterstaining with hematoxilin. Immunofluorescence analysis of brain sections for parasite sequestration was carried out by fixing the brain samples in 4% paraformaldehyde in PBS containing 20% sucrose for 24 h at 4° C. and cryoprotecting them for 48 h in PBS containing 20% sucrose. Coronal sections of 30 μm thickness was prepared using cryostat microtome and antigen retrieval was carried out by treating them at 95° C. for 30 min in sodium citrate buffer pH 9.0. After blocking with 1% BSA, the sections were incubated with anti-CD31 mouse monoclonal antibody conjugated with Alexa Fluor 594 and anti-PbGAPDH rabbit polyclonal serum or anti-mouse CD3 rat monoclonal antibody for 16 h at 4° C. The sections were then treated with FITC-conjugated donkey anti-rabbit IgG or FITC-conjugated goat anti-rat IgG (1:200 dilution), followed by 4′,6-diamidino-2-phenylindole (DAPI) staining. Anti-mouse β-APP rabbit polyclonal antibody was used in 1:200 dilution.

### Example 3

Heme, Hz, hemoglobin and hemopexin estimations. Free heme levels in the plasma samples of WT- and KO-infected mice were quantified using Hemin colorimetric assays. The assay is specific for free heme and it utilizes peroxidase activity of hemin to facilitate the conversion of a colorless probe to a strongly colored compound with absorbance at 570 nm. The quantification of free heme in the parasite lysates was carried out by resuspending the parasite pellets in 5 volumes of hypotonic lysis buffer containing 5 mM Tris pH 7.5 with protease inhibitors and incubating them in ice for 30 min. The lysates were then centrifuged at 20,000 g for 20 min, 4° C., and the supernatants obtained were used for free heme estimation as mentioned above for the plasma samples. The Hz content of the WT and KO parasite pellets was estimated as described. The parasite pellet was resuspended in 1 ml of 100 mM sodium acetate buffer, pH 5.0 and left at 37° C. for overnight, followed by centrifugation at 10,000 g for 5 min. The resultant pellet was resuspended in 100 mM Tris buffer pH 8.0 containing 2.5% SDS and incubated at 37° C. for 30 min, followed by centrifugation at 10,000 g for 5 min. The pellet obtained was washed once with 100 mM alkaline bicarbonate pH 9.2 and then with distilled water. The final Hz pellet was dissolved in 100 mM NaOH containing 2.5% SDS, and the absorbance was measured at 405 nm. The supernatants of sodium acetate and Tris SDS steps were collected to estimate the protein content by Micro BCA protein assay kit and heme content of the Hz was expressed per mg of total protein. To estimate the Hz content in the spleen and liver of the WT- and KO-infected mice, 50 mg tissue of the respective organs was homogenized in 50 mM Tris pH 8.0 containing 50 mM NaCl, 5 mM CaCl₂ and 1% Triton X-100, and incubated for 12 h at 37° C. in the presence of proteinase K. The lysates were sonicated and centrifuged at 15,000 g for 30 min. The pellets obtained were resuspended with 100 mM sodium bicarbonate containing 2% SDS and sonicated, followed by centrifugation at 15,000 g for 15 min. After repeating this step thrice, the pellets were solubilized in 100 mM NaOH containing 2% SDS and 3 mM EDTA, and the absorbance was measured at 405 nm. In parallel, the parasite load in the organs was examined by quantitative PCR (qPCR) analysis carried out with Pb18SrRNA primers for the total RNA isolated from 30 mg tissue of the organs. After normalizing with respect to the parasite load, the total heme content of the Hz isolated was expressed per mg weight of the organ. Hemoglobin and hemopexin levels in the plasma samples of WT- and KO-infected mice were measured by ELISA.

### Example 4

In vivo bioluminescence imaging. In vivo bioluminescence imaging for mice infected with WT and KO parasite lines expressing luciferase was carried out as described. Luc-expressing ALASKO and FCKO parasites were generated by transfecting WT parasites with GOMO-GFP-Luc plasmid containing GFP-Luc-expressing cassette with m-cherry flanked on either side by 5′- and 3′-UTR regions of the respective genes. For whole body imaging, 7-8 weeks old C57BL/6 mice were injected with Luc-expressing 10⁵ WT or 10⁷ KO parasites. On day 8 post-infection, mice were injected intraperitoneally with D-luciferin substrate (100 mg/kg animal weight in 200 μl of PBS), and imaged after 5 min using in vivo Imaging System with medium binning, 10 sec exposure and 12.5 FOV, under gas anesthesia system. For ex vivo imaging, transcardial perfusion was carried out for infected mice with cold PBS after injecting D-luciferin, and the organs were dissected out and imaged.

### Example 5

Analyses of cytokines, chemokines, chemokine receptors and other key mediators of cerebral pathogenesis. Bio-Plex assays for cytokines and chemokines were carried out. The plasma samples utilized for the assays were prepared from the infected mouse blood collected on day 7/8 post-infection. For transcript levels, total RNA was isolated from the brain samples of WT and KO-infected mice that were collected after a thorough perfusion with cold PBS and qPCR analyses were performed. Expression levels were normalized with GAPDH and fold changes for the transcripts of KO-infected mice with respect to WT were calculated using 2^{(-ΔΔCt)} method.

### Example 6

Flow cytometry. Flow cytometry analyses of T cells in the brain samples of WT- and KO-infected mice were carried out as described. Mice were anesthetized and transcardially perfused with PBS, and the brain samples were dissected out and harvested in RPMI-1640 medium containing 10% FBS. For preparing single cell suspensions, the samples were minced and digested in RPMI-1640 medium containing 0.05% Collagenase D and 2 U/ml DNase I for 30 minutes at room temperature, and passed through 70 μm nylon cell strainer, followed by 5 minutes of incubation on ice. Brain homogenates were then overlaid on 30% Percoll cushion and centrifuged at 400 g for 20 minutes at room temperature. The leukocyte pellets obtained were resuspended in 1 ml of RBC lysis buffer (155 mM NH₄Cl, 10 mM NaHCO₃ and 0.1 mM EDTA; pH 7.3) and incubated on ice for 5 minutes to remove any residual RBCs. The pellets were then washed with RPMI-1640, counted and stained for various markers. For intracellular markers like TNFα, IFNγ, perforin and granzyme B, staining was carried out after fixing the cells with 4% paraformaldehyde.

### Example 7

Labelling studies with ¹⁴C-ALA. The infected blood samples were collected, centrifuged at 1,000 g for 5 min to remove plasma and buffy coat, and washed twice with RPMI-1640 medium containing 10% FBS. The washed cells were resuspended in 10 volumes of RPMI-1640 medium containing 10% FBS and then incubated at 37° C. in a CO₂ incubator with the respective radioactive compounds. For ¹⁴C-ALA labelling, blood samples were collected from griseofulvin treated and control WT-infected mice around 16:00 h and the labelling was carried out for 9 h at a radioactivity of 1 Ci/ml. The infected RBCs were then centrifuged, washed with PBS and the parasites were isolated by saponin treatment. After washing the parasite pellet with PBS for four times, free heme present in the parasites was extracted using ethylacetate:glacial acetate (4:1) followed by 1.5 N HCl and water washes to remove porphyrins and ALA. The upper phase was separated, dried under nitrogen stream, dissolved in methanol and analysed by TLC on silica gel using the mobile phase 2,6-lutidine and water (5:3 v/v) in ammonia atmosphere. The intensity of radiolabelling was scanned using Biomolecular Imager by exposing the TLC sheets to phosphorimager screen and the radioactive counts were measured.

### Example 8

Griseofulvin treatment in mice. Griseofulvin was prepared by dissolving 1 or 2 mg in 40 μl DMSO and then making up the volume to 200 μl with 10% solutol HS 15 in saline. The mixture was vortexed thoroughly for 10 min to form an emulsion and injected intraperitoneally into the mice. All single dose injections were carried out at 06:00 h and double dose injections were carried out at 06:00 h and 18:00 h for the respective days. Control mice were injected with the solvent. α,β-arteether (E MAL) was administered intramuscularly. For experiments involving the combination of α,β-arteether and griseofulvin, α,β-arteether was administered intramuscularly at 06:00 h on day 6, followed by two doses of griseofulvin at 24 h interval (at 09:00 h on day 6 and 7).

The present invention explored the possibility of including griseofulvin in the treatment protocol in affected patient population. The only inclusion will be griseofulvin for three days to prevent the development of cerebral and severe malaria. In humans, griseofulvin dosage is given to the extent of 500-1000 mg/day in adults and 5-10 mg/kg/day in children for several weeks. The conversion of 5-10 mg/kg/day into animal dose is carried out by multiplying human dose with 12.3. Taking the average weight of 7-8 weeks mouse as 25 g, this comes to 1.5-3.0 mg per mouse. Therefore, the dose used for griseofulvin (2 mg per mouse per day) in this invention is matching to human dose that has been routinely used for the treatment of fungal infections.

Griseofulvin would be included in the treatment protocol for three days at the same dosage (500-1000 mg/day in adults and 5-10 mg/kg/day for children) that has been used for fungal diseases along with the existing ACTs to prevent cerebral and severe malaria. The treatment protocol suggested in this invention will be the standard ACT that has been given as per the WHO recommendation for malaria patients along with griseofulvin for three days starting from the first day of ACT treatment. There will not be any change in the standard malaria therapy except that the griseofulvin will be included for three days. The ACTs recommended by WHO are α,β-arteether/artemether in combination with lumefantrine or artesunate in combination with amodiaquine or artesunate in combination with mefloquine or dihydroartemisinin in combination with piperaquine or artesunate in combination with sulfadoxine-pyrinethamine. This ACT treatment is given for 3 days.

Now, the crux of the invention is claimed implicitly and explicitly through the following claims. Each of the appended claims defines a separate invention, which for infringement purposes is recognized as including equivalents to the various elements or limitations specified in the claims. Depending on the context, all references below to the “invention” may in some cases refer to certain specific embodiments only. In other cases, it will be recognized that references to the “invention” will refer to subject matter recited in one or more, but not necessarily all, of the claims. Groupings of alternative elements or embodiments of the invention disclosed herein are not to be construed as limitations. Each group member can be referred to a claimed individually or in any combination with other members of the group or other elements found herein. One or more members of a group can be included in or deleted from, a group for reasons of convenience and/or patentability. When any such inclusion or deletion occurs, the specification is herein deemed to contain the group as modified thus fulfilling the written description of all groups used in the appended claims. It is to be noted that the present invention is susceptible to modifications, adaptations and changes by those skilled in the art. Such variant embodiments employing the concepts and features of this invention are intended to be within the scope of the present invention, which is further set forth under the following claims.

We claim:

1. A pharmaceutical composition against cerebral and severe malaria comprising artemisinin-based combination therapy (ACT), in combination with griseofulvin, for three days.

2. The composition as claimed in claim 1, wherein 500-1000 mg/day of griseofulvin is used in adults and 5-10 mg/kg/day of griseofulvin in children, in combination with ACT.

3. The composition as claimed in claim 1, characterized in that, the ACTs that are recommended are α,β-arteether in combination with lumefantrine or artemether in combination with lumefantrine or artesunate in combination with amodiaquine or artesunate in combination with mefloquine or dihydroartemisinin in combination with piperaquine or artesunate in combination with sulfadoxine-pyrimethamine.

4. A method of preventing and treating cerebral and severe malaria comprising administering of griseofulvin in combination with artemisinin-based combination therapy (ACT), for three days.

5. The method as claimed in claim 4, wherein 500-1000 mg/day of griseofulvin is used in adults and 5-10 mg/kg/day of griseofulvin in children, in combination with ACT.

6. The method as claimed in claim 4, wherein the ACTs that are recommended are α,β-arteether in combination with lumefantrine or artemether in combination with lumefantrine or artesunate in combination with amodiaquine or artesunate in combination with mefloquine or dihydroartemisinin in combination with piperaquine or artesunate in combination with sulfadoxine-pyrimethamine.

7. Griseofulvin for use in a method of prevention or treatment of cerebral or severe malaria, in combination with artemisinin-based combination therapy (ACT).

8. Griseofulvin for use in a method of prevention or treatment of cerebral or severe malaria as claimed in claim 7, wherein 500-1000 mg/day of griseofulvin is used in adults and 5-10 mg/kg/day of griseofulvin in children in combination with ACTs.

9. Griseofulvin for use in a method of prevention or treatment of cerebral or severe malaria as claimed in claim 7 and 8, wherein the ACTs that are recommended are α,β-arteether in combination with lumefantrine or artemether in combination with lumefantrine or artesunate in combination with amodiaquine or artesunate in combination with mefloquine or dihydroartemisinin in combination with piperaquine or artesunate in combination with sulfadoxine-pyrimethamine.
